(19) United States
(12) Patent Application Publication
(10) Pub. No.: US 2026/0108253 A1
(43) Pub. Date: Apr. 23, 2026

(54) MECHANICALLY TAILORED EXPANSION OF SHAPE MEMORY FOAM

(71) Applicant: BOSTON SCIENTIFIC SCIMED, INC., MAPLE GROVE, MN (US)

(72) Inventors: Patrick Willoughby, Shoreview, MN (US); Scott Andrew McCullough, Plymouth, MN (US); Niraj Gurung, Monticello, MN (US); Joshua Mark Inouye, Brooklyn Park, MN (US)

(73) Assignee: BOSTON SCIENTIFIC SCIMED, INC., MAPLE GROVE, MN (US)

(21) Appl. No.: 19/365,830

(22) Filed: Oct. 22, 2025

Related U.S. Application Data
(60) Provisional application No. 63/710,716, filed on Oct. 23, 2024.

(51) Int. Cl. A61B 17/12; A61B 17/00

(52) U.S. Cl. CPC A61B 17/1219; A61B 17/12122; A61B 2017/00871; A61B 2017/1205

(57) ABSTRACT

An expandable foam implant includes an element formed from a shape memory polymer and radially crimped, where the radially crimped element has an axial or circumferential outer surface of tightly packed pores resistant to liquid ingress. The shape memory polymer element includes a surface treatment disposed on at least a first portion of the axial or circumferential outer surface, where liquid ingress into the first portion is faster than into a remainder of the axial or circumferential outer surface of the radially crimped element.

### CROSS-REFERENCE TO RELATED APPLICATIONS

This application claims the benefit of priority of U.S. Provisional Application No. 63/710,716 filed Oct. 23, 2024, the entire disclosure of which is hereby incorporated by reference.

### TECHNICAL FIELD

The disclosure pertains to medical devices and more particularly to medical devices incorporating a shape memory foam component.

### BACKGROUND

A wide variety of medical devices have been developed for medical use including, for example, medical devices implanted within the heart that are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example expandable foam implant includes an element formed from a shape memory polymer foam and radially crimped, the radially crimped element having an axial or circumferential outer surface of tightly packed pores resistant to liquid ingress, and a surface treatment disposed on at least a first portion of the axial or circumferential outer surface, wherein liquid ingress into the first portion is faster than into a remainder of the axial or circumferential outer surface of the radially crimped element.

Alternatively or additionally to any of the embodiments above, the surface treatment includes a plurality of shaved, scored, or abraded regions extending along an outer surface of the crimped element.

Alternatively or additionally to any of the embodiments above, the plurality of shaved, scored, or abraded regions includes a plurality of axially extending abraded stripes spaced apart around a circumference of the crimped element.

Alternatively or additionally to any of the embodiments above, including 15-20 axially extending abraded stripes each measuring 1.0 mm to 2.0 mm in a circumferential direction.

Alternatively or additionally to any of the embodiments above, the plurality of shaved, scored, or abraded regions includes a plurality of helical grooves.

Alternatively or additionally to any of the embodiments above, the plurality of shaved, scored, or abraded regions cover between 25% and 50% of the axial or circumferential outer surface.

Alternatively or additionally to any of the embodiments above, the surface treatment includes a temporary agent disposed within pores in the foam before being radially crimped, the temporary agent configured to be dissolved when the element is exposed to liquid.

Alternatively or additionally to any of the embodiments above, the temporary agent includes at least one of a polymer, salt, or sugar.

Alternatively or additionally to any of the embodiments above, areas of the foam devoid of the temporary agent expand before areas with the temporary agent.

Alternatively or additionally to any of the embodiments above, the temporary agent has a removal time that is tuned to achieve a desired expansion profile.

Alternatively or additionally to any of the embodiments above, areas of the element with the temporary agent expand more quickly than areas without the temporary agent when the temporary agent has a short removal time.

Alternatively or additionally to any of the embodiments above, the expandable foam implant further includes an internal permeable lumen within the crimped element.

Alternatively or additionally to any of the embodiments above, the internal permeable lumen is configured to allow application of negative pressure to draw liquid into the element or positive pressure to inject liquid directly into the interior of the element.

Alternatively or additionally to any of the embodiments above, the internal permeable lumen is formed by a permeable polymer tube.

Another example expandable foam implant includes an element including at least first and second regions of open-celled foam, and at least a first barrier region separating the first and second regions, the first barrier configured to restrict liquid flowing between the first and second regions, wherein liquid ingress into the first region is faster than into the second region.

Alternatively or additionally to any of the embodiments above, the first region is a cylindrical shell and the second region is a cylindrical core surrounded by the first region.

Alternatively or additionally to any of the embodiments above, the expandable foam implant further including a third region, wherein the first region defines a distal end of the element, the second region defines a middle region of the element, and the third region defines a proximal region of the element, wherein a first barrier is disposed between the first and second regions and a second barrier is disposed between the second and third regions, wherein the first region is configured to expand first when the element is exposed to liquid, followed by the second region, with the third region expanding last.

An example method of controlling expansion of a shape memory foam implant includes constraining a crimped shape memory foam element within a catheter such that only a distal face of the element is exposed, allowing liquid ingress to occur at the exposed distal face during procedural steps, and fully deploying the element from the catheter to achieve apparent instant expansion.

Alternatively or additionally to any of the embodiments above, the procedural steps comprise at least one of flushing, translation through an access sheath, or dwelling within a body.

Alternatively or additionally to any of the embodiments above, the method further includes creating holes in the catheter to allow controlled liquid ingress prior to full deployment.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIGS. 1A and 1B illustrate a crimped shape memory polymer foam element in an unexpanded and partially expanded configuration, respectively;

FIGS. 1C and 1D illustrate another crimped shape memory polymer foam element in an unexpanded and partially expanded configuration, respectively;

FIGS. 2A-2D illustrate crimped shape memory polymer foam elements with various surface treatments expanding from an unexpanded configuration to a fully expanded configuration;

FIG. 3 illustrates a crimped shape memory polymer foam element with a wire constraining one end;

FIGS. 4A-4C illustrate various shape memory polymer foam elements formed from multiple sections separated by a barrier;

FIGS. 5A-5C illustrate a crimped shape memory foam element with a central lumen expanding under negative pressure;

FIGS. 6A-6C illustrate a crimped shape memory foam element with a central lumen expanding under positive pressure;

FIGS. 7A-7C illustrate a crimped shape memory foam element with a central lumen expanding under negative pressure and then a central cavity being filled; and

FIGS. 8A and 8B illustrate a method of rapid expansion of a crimped shape memory foam element from a delivery catheter.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same”shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

A left atrial appendage closure (LAAC) device may be adapted for occluding a patient's left atrial appendage (LAA). The LAAC device may include a shape memory polymer (SMP) foam element that is adapted to expand from a crimped configuration for delivery to an expanded configuration after delivery into the LAA. The SMP foam element may have a first, compressed, profile when in its crimped configuration and may have a second, expanded, profile different from the first profile when in its expanded configuration. The second, expanded, profile may include a diameter greater than a diameter in the compressed profile. The length of the SMP foam element may be substantially the same in the first and second profiles, or the length in the first profile may be greater or shorter than in the second profile. The SMP foam element includes in its compressed, crimped configuration a distal face, a proximal face, and an axial or circumferential outer surface.

Crimping is a common technique used to prepare SMP foam elements for delivery and implantation. The crimping process involves compressing the foam element into a smaller, more manageable shape that can be easily inserted through a catheter or other delivery device. During the crimping process, the SMP foam element may be initially in its expanded or memorized state, and then radial and/or axial constraints are applied to transform the SMP foam element from its expanded state to its temporary state. In some cases, the crimping process may be performed at elevated temperatures to facilitate compression or to influence the foam element's expansion characteristics. To achieve good shape memory behavior, the SMP foam element is crimped at a temperature above the dry glass transition temperature (Tg) of the polymer to obtain an elastic response that can be properly recovered by a later temperature exposure. Crimping below the Tg may induce an amount of plastic deformation which is non-recoverable without a high temperature pseudo-melt type process. Below the Tg, the material acts like a hard, glassy, plastic material. Above the Tg the material acts like a soft rubbery material. Shape is held by elastically deforming above the Tg when it is soft and squishy, then cooling below the Tg so that the SMP foam element remains in the new configuration because it is stiffer. These properties are true for many polymer systems that are amorphous or semi-crystalline. A distinction for the SMP materials desired for the SMP foam elements is that they have a different Tg when dry than when wet. The dry Tg is designed to be higher than the use temperature of 37° C., so the crimped element will not expand when warm. When exposed to fluid, the Tg is lowered. The wet Tg is designed to be less than the use temperature of 37° C., so the crimped element will expand when warm and wet.

Crimping SMP foam elements can alter their surface pore structure. This is primarily due to the mechanical compression involved in the process. Compression-induced pore collapse may result in the closure of some pores. As the SMP foam element is compressed during crimping, the pressure can force smaller pores to close, reducing their overall surface area. Larger pores may become deformed or flattened, altering their shape and potentially reducing their effective size. The crimping process may also cause some pores to become more aligned, especially in the direction of compression. This may affect the foam element's permeability and the ability of liquids to enter the pores. The crimping process may disrupt the connectivity between pores, creating isolated pockets or dead-end pores. This may impact the SMP foam element's fluid transport properties. The amount of force and pressure applied during crimping can also influence the degree of pore deformation and closure.

The properties of the SMP foam element material, such as its elasticity and pore size distribution, may also affect how the pores respond to compression. Changes in pore structure may alter the SMP foam element's permeability, affecting its ability to absorb or release liquids. The element's expansion behavior after crimping may be affected by changes in pore structure, as pores can act as nucleation sites for expansion. Materials with a fixed Tg have a limited change in expansion due to the liquid ingress alone, but will expand more quickly due to the thermal capacity of the liquid. For example, cold liquid ingress will not cause expansion by itself and may act to slow down expansion by cooling the SMP foam element, while warm liquid ingress will speed up expansion by warming up the foam more quickly. For materials that have mechanisms like solvent based plasticization or easily broken hydrogen bonding, the water itself will directly change the expansion characteristic by decreasing the Tg. Ingress of liquid below the wet Tg will depress the Tg, but not induce physical expansion. Liquid above the wet Tg will depress the Tg and induce physical expansion.

The above changes in SMP pore structure may result in a crimped SMP foam element used as an LAAC that is resistant to liquid absorption, and/or has uneven liquid absorption, which may lead to an inconsistent and/or unexpected expansion when exposed to moisture and liquids in the body during and after implantation. To control or improve liquid absorption and expansion, one or more surfaces of the crimped SMP foam element, including the distal and proximal faces, may be altered by shaving, cutting, scoring, slitting, abrading, and/or incising. The crimped SMP foam element is mechanically altered when in a cold and dry state. The crimped axial or circumferential outer surface of the SMP foam element have tightly bound pores, which reduces the rate of liquid uptake, while the altered surfaces on the distal and proximal ends of the SMP foam element have more open and direct access into the pores, increasing liquid uptake and causing faster foam expansion on the altered surface ends.

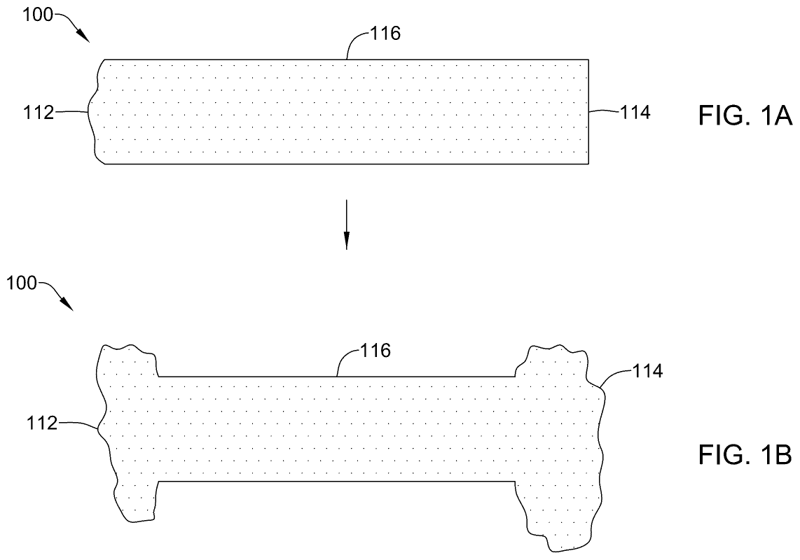

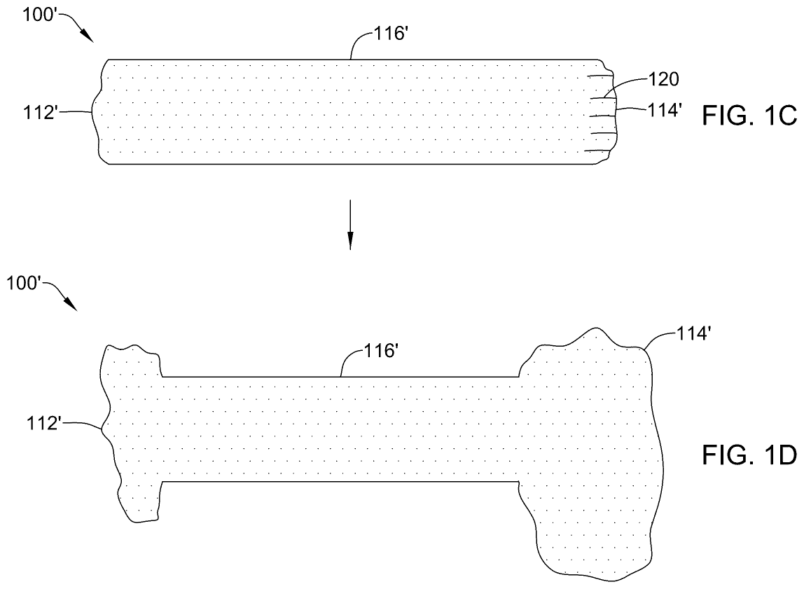

FIG. 1A illustrates a crimped SMP foam element 100 with the distal face 114 cut straight across to expose open pores on the distal face 114, while the proximal face 112 and axial or circumferential outer surface 116 are unmodified, maintaining a compressed configuration with tightly bound pores. FIG. 1B illustrates the crimped SMP foam element 100 after being submerged in liquid at 37° C. for less than two minutes. The liquid may be water or blood. The cut distal face 114 has opened the pores of the foam element 100 resulting in expansion with the influx of liquid axially into the open pores through the cut face 114, while the unmodified proximal face 112 shows modest expansion due to limited liquid infiltration axially into the unmodified pores and the axial or circumferential outer surface 116 extending between the proximal and distal faces 112, 114, remains in the compressed configuration because liquid has not been able to infiltrate radially into the pores along the crimped outer surface.

FIG. 1C illustrates a crimped SMP foam element 100′ similar to SMP foam element 100, but with the distal face 114′ having a plurality of surface cuts or abrasions 120. The plurality of surface cuts or abrasions 120 may be positioned only on the distal face 114′ or they may extend over a portion of the axial or circumferential outer surface 116′ as illustrated. FIG. 1D illustrates the crimped SMP foam element 100′ after being submerged in liquid at 37° C. for less than two minutes. The liquid may be water or blood. The surface cuts or abrasions 120 on the distal face 114′ have opened the pores of the foam element 100′ resulting in expansion with the influx of liquid axially and radially into the open pores through the surface cuts or abrasions 120, while the unmodified proximal face 112′ shows modest expansion due to limited liquid infiltration axially into the unmodified pores and the majority of the axial or circumferential outer surface 116′ remains in the compressed configuration because liquid has not been able to infiltrate radially into the pores along the crimped outer surface.

In some embodiments, the SMP foam element may have a plurality of different surface treatments in different regions to achieve a tailored expansion rate of rapid expansion in some regions and slower expansion in other regions. In one embodiment, the proximal end may have a surface treatment configured to achieve rapid expansion to occlude the LAA quickly, while having a reduced profile of the overall device to facilitate removal or repositioning if needed.

In some embodiments, a surface treatment may also be disposed on at least a first portion of the axial or circumferential outer surface 116. The following surface treatments may result in liquid ingress into the first portion being faster than ingress into a remainder of the axial or circumferential outer surface 116 of the radially crimped foam element.

One embodiment of a surface treatment is the mechanical removal of material from the outer surface of the crimped SMP foam element implant. Mechanical removal may include shaving, scoring, or otherwise abrading the outer surface to form shallow grooves or abrasions on the outer surface of the crimped SMP foam element. Abrading may be achieved with a rasp or other roughening tool. The removal of a shallow layer of material from the outer surface of the crimped SMP foam element 100 may expose the inner open cell structure and allow for faster radial liquid ingress and expansion as compared to the tightly packed surface pores created during crimping.

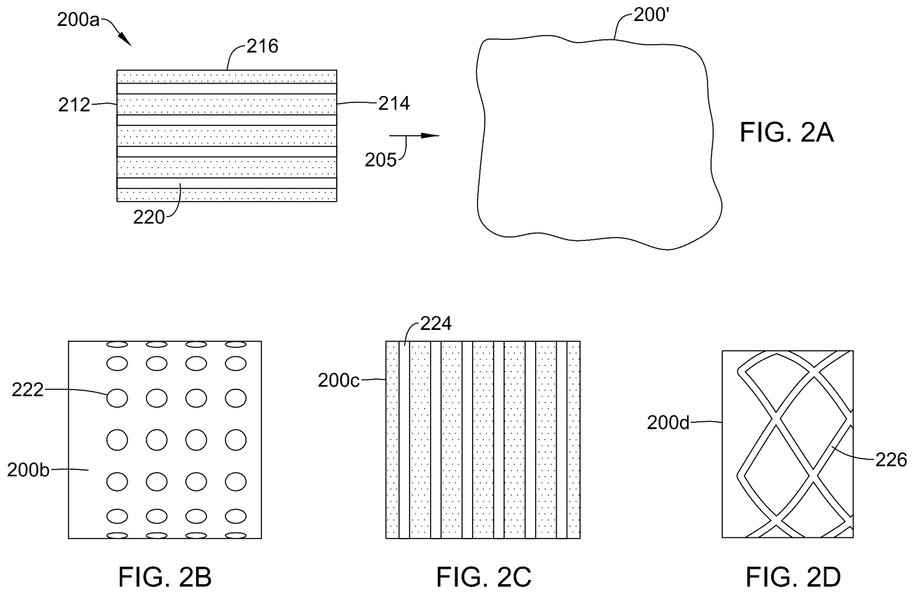

FIG. 2A illustrates an elongate crimped SMP foam element 200a with a distal face 214, a proximal face 212, and an axial or circumferential outer surface 216 extending therebetween. The crimped SMP foam element 200a may include a plurality of regions 220 in which at least the outer surface 216 of the foam element has been removed or disrupted. For example, regions 220 in the outer surface 216 may be shaved, scored, or abraded. The plurality of shaved, scored, or abraded regions 220 may provide a higher surface area compared to the remainder of the crimped outer surface 216, which may increase ingress of liquid radially into the foam and decrease expansion time. The plurality of shaved, scored, or abraded regions 220 may extend over the entire length of the SMP foam element 200a or they may be defined as one or more discrete regions. The plurality of shaved, scored, or abraded regions 220 may extend completely around the circumference of the element, or just a portion of the circumference.

In the embodiment shown in FIG. 2A, the shaved, scored, or abraded regions 220 are in the form of a plurality of axially extending stripes 220 extending from the distal face 214 to the proximal face 212. The stripes 220 may be spaced apart around the circumference of the foam element 200a, with 3 to 25 stripes spaced apart circumferentially around the SMP foam element 200a. In some embodiments 15-20 axially extending stripes 220 may be spaced apart circumferentially around the crimped SMP foam element 200a. In one example, 18 circumferentially spaced apart stripes 220 may be formed in the surface of the crimped SMP foam element 200a. In the expanded configuration, the SMP foam element 200a may have any shape, and may have a diameter and/or length of between 10 millimeters (mm) (0.39 inches) and 50 mm (1.97 inches). The stripes 220 may each have a width measuring 0.5 mm to 4 mm. In some embodiments, the stripes 220 may measure 1.0 mm to 2.0 mm in the circumferential direction. The stripes 220 may cover at least 10% to 50% of the surface of the crimped SMP foam element 200a. In other embodiments, up to 75% of the surface of the crimped SMP foam element 200a may be covered by stripes 220. Additionally, in some embodiments, the entire outer surface of the crimped SMP foam element 200a may be covered by surface cuts and/or abrasions.

In some embodiments, the plurality of shaved, scored, or abraded regions 220 may extend radially into the axial or circumferential outer surface 216 of the crimped SMP foam element 200a just far enough to disturb the surface structure, for example to a depth of 75 micrometers (μm) to 125 μm. In other embodiments, the plurality of shaved, scored, or abraded regions 220 may extend radially into the axial or circumferential outer surface 216 of the crimped SMP foam element 200a to a depth matching the expanded pore size to ensure full ingress of liquid into the structure as it expands, such as to a depth of 0.3 mm to 2 mm. In further embodiments, a hole at or above the pore size may extend all the way through the SMP foam element or stop at the center line. Helical, opposing helical, or straight cuts would not be able to go all the way through without turning into multiple parts. A limit on the depth of such cuts may be no more than 50% of the SMP foam element diameter for a foam-only design. In some embodiments, a solid core may be included (e.g. a machined steel rod), in which case the cuts may extend to the solid core as it would connect and retain the component parts of the SMP foam element. In a further embodiment the entirety of the axial or circumferential outer surface 216 may be shaved, scored, or abraded.

When the crimped SMP foam element 200a is exposed to liquid, represented by arrow 205, the plurality of shaved, scored, or abraded regions 220 allow for the rapid radial ingress of liquid, resulting in a faster expansion of the foam element 200a to the expanded state 200′, where the expansion is targeted to the area with the shaved, scored, or abraded regions 220. In some embodiments, the orientation and pattern of the shaved, scored, or abraded regions 220 may provide substantially uniform expansion of the SMP foam element 200a. The expansion may occur faster and more uniformly that the crimped SMP foam element 100 with only the surfaces of the distal and proximal faces cut or abraded, illustrated in FIG. 1B. In addition to the shaved, scored, or abraded axial stripes 220 shown in FIG. 2A, the plurality of shaved, scored, or abraded regions may be discrete holes 222 extending radially into the foam element, circumferential stripes 224, or opposing helical stripes 226 shown on the crimped SMP foam elements 200b, 200c, 200d, shown in FIGS. 2B, 2C, and 2D, respectively. The opposing helical stripes 226 may include a first continuous helical stripe extending in a first direction and a second continuous helical stripe extending in the opposite direction. Additionally, any combination of all of the configurations of shaved, scored, or abraded regions shown in FIGS. 2A-2D may be used. In all configurations, when the crimped SMP foam elements 200b, 200c, 200d are in the presence of liquid, relatively uniform, rapid expansion results in the fully expanded SMP foam element 200′. In addition to shaving, scoring, and abrading the outer surface of the crimped SMP foam element, a laser may be used to provide surface disruption. For example, a laser may be used to drill the plurality of discrete, spaced apart holes 222 shown in FIG. 2B, to form the stripes 220, 224 shown in FIGS. 2A and 2C, and to form the opposing helical stripes or grooves 226 shown in FIG. 2D.

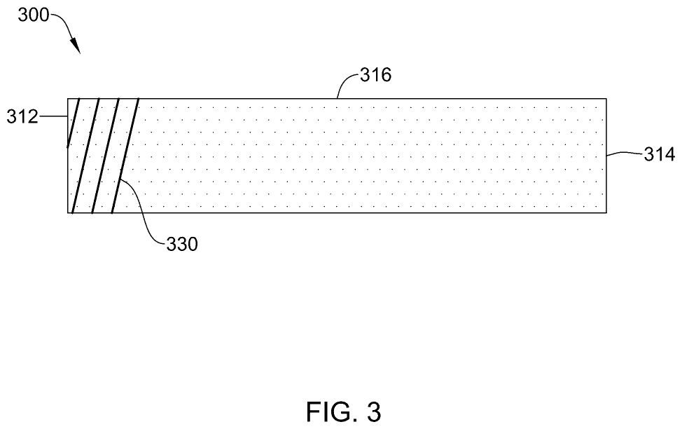

In another embodiment, a surface treatment may be achieved by adding a constrainment member to physically constrain at least a portion of the crimped SMP foam element. The constrainment member may prevent, delay, or reduce liquid ingress radially into the foam cell structure in the constrained locations, while allowing liquid ingress into the regions between constrained locations. FIG. 3 illustrates a crimped SMP foam element 300 with an unconstrained distal face 314 and axial or circumferential outer surface 316. A constrainment member 330 may be disposed circumferentially around a first end region of the crimped SMP foam element 300. In the embodiment shown in FIG. 3, the constrainment member 330 is a ribbon or wire 330 wrapped helically around the proximal end 312 of the SMP foam element 300. In some embodiments, wraps of the ribbon or wire 330 may be spaced apart, as shown in FIG. 3, which may allow liquid ingress radially into the foam structure between the wraps of ribbon or wire 330 while preventing liquid ingress in the region directly under the ribbon or wire 330. In other embodiments, the wraps of the ribbon or wire 330 may abut one another to form a substantially solid constrainment structure. In still further embodiments, the constrainment member 330 may be a solid band or tube disposed circumferentially around the SMP foam element 300.

In a further embodiment, the surface treatment may include a solution of a temporary agent disposed within the pores of the SMP foam element before being radially crimped. The temporary agent is configured to reduce or prevent liquid ingress radially into the foam cell structure while it is present, but to be dissolved when the foam element is exposed to liquid. The temporary agent may be applied before the SMP foam element is crimped and may be configured to dissolve or be washed out of the foam cell structure when the treated and crimped SMP foam element is exposed to liquid. The placement, type and amount of the temporary agent applied to the SMP foam element may control the rate of expansion of the foam element when exposed to liquid. Examples of solutions of temporary agents are water soluble and include polyethylene glycol (PEG), polylactic acid (PLA), poly-l-lactic acid (PLLA), salts, or sugars. The temporary agent may also include a drug to be delivered to the body upon implantation and/or a marker or contrast agent. The solution of temporary agent may be sprayed onto the surface of the uncrimped SMP foam element, the element may be dipped into the solution, or the solution may be drawn into the pores, such as by compressing the foam element and releasing the element while submerged in the solution of temporary agent. The SMP foam element with incorporated temporary agent would then be crimped and processed as normal. The packaged and sterilized crimped SMP foam element would remain in the crimped configuration during normal shipping conditions.

At the time of implantation into the body, the treated crimped SMP foam element would be exposed to a liquid such as water or blood. This liquid would dissolve or wash away the temporary agent, exposing and opening the pore structure of the crimped SMP foam element. In other embodiments, the treated and crimped SMP foam element may be washed as a prep step, during a flushing step, during catheter transit, or during dwelling inside the blood or anatomy. Moisture and/or liquid would immediately enter the freshly exposed foam element cellular structure, resulting in expansion of the foam element. The temporary agent may have a removal and/or dissolving time that may be selected, such as with the amount and/or placement of the temporary agent on the SMP foam element, to achieve a desired expansion profile. For example, if the temporary agent has a long removal time (e.g. (PLLA), then areas of the foam element without the temporary agent will expand first and the areas containing the temporary agent would expand when the temporary agent is removed or when moisture ingresses around the temporary agent. If the temporary agent has a short removal time (e.g. salt), then areas of the foam element containing the temporary agent will expand more quickly than areas without the temporary agent. This is due to the crimped surface of the foam element being resistant to liquid ingress, whereas the pores in regions containing the temporary agent are open once the temporary agent is removed, and readily accept liquid ingress. In some embodiments, the foam element may include a combination of surface treatments (i.e., the distal face, proximal face or axial or circumferential outer surface may be cut or abraded, and a temporary agent may also be disposed on the faces or outer surface).

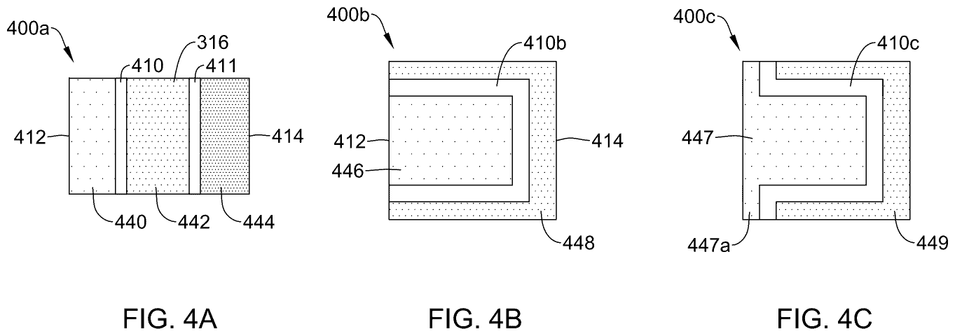

In addition to the surface treatments discussed above, another structure that may be used to alter the rate and/or location of liquid ingress into a foam element is to include one or more barriers into a composite open cell foam element construction. The barriers may be partially or fully occlusive to liquid and may separate regions of open cell foam having the same or different cellular structure. FIGS. 4A, 4B, and 4C illustrate different combinations of barriers and foam regions forming expandable foam element implants 400a, 400b, 400c. Each expandable foam implant 400a, 400b, 400c may be formed from one or more foam regions. In FIG. 4A, a first foam region 440 may form the proximal face 412 of the foam element implant 400a. A first barrier 410 may separate the first foam region 440 from a second foam region 442, and a second barrier 411 may separate the second foam region 442 from a third foam region 444 which forms the distal face 414 of the foam element implant 400a. The first barrier 410 and the second barrier 411 may have different levels of liquid permeability. Each of the foam regions 440, 442, 444 may allow liquid to flow easily through the open celled foam, but liquid cannot communicate as easily across the first and second barriers 410, 411. The first, second, and third foam regions 440, 442, 444 may all have the same liquid permeability, or each may have a different liquid permeability. In some embodiments, the first foam region 440 may have the lowest liquid permeability followed by the second foam region 442, and then the third region 444. The first and second barriers 410, 411 may be impermeable to liquids, such that the only liquid ingress into the second foam region 442 is radially through the outer surface 416 such that the second foam region 442 may expand much more slowly than either or both regions 440, 444. In other embodiments, the first and second barriers 410, 411 may have different liquid permeabilities. When the first and/or second barrier 410, 411 is permeable to liquid, liquid may enter into the second foam region 442 radially from the outer surface 416 and axially from one or both of the first and third foam regions 440, 444. The first foam region 440 and the third foam region 444 may allow liquid ingress axially and radially into the foam structures. When the foam element implant 400a is implanted in the body, the third foam region 444 may form a distal face 414 of the implant 400a, and may expand earlier than the second foam region 442, followed by the first foam region 440. In other embodiments, the direction of expansion may be reversed, with the first foam region 440 expanding first, followed by the second foam region 442 and then the third foam region 444.

In the embodiment illustrated in FIG. 4B, a cylindrical central core foam region 446 may be surrounded circumferentially and at a distal end by a barrier 410b. The central core foam region 446 may form the proximal face 412 of the foam implant 400b. An outer cylindrical shell 448 may be disposed circumferentially around the barrier 410b and may form the distal face 414 of the foam implant 400b. The barrier 410b may be impermeable to liquid such that liquid enters the central core foam region 446 only axially from the proximal face 412. In other embodiments, the barrier 410b may be permeable to liquid such that liquid may enter the central core foam region 446 from both the proximal face 412 and the outer shell 448. The central core foam region 446 may have a lower permeability than the outer shell 448 such that the outer shell 448 may expand first, followed by the central core 446. In other embodiments, the central core foam region 446 may have a higher permeability than the outer shell 448 such that when the foam element implant 400b is deployed in the body, the central core foam region 446 may expand quickly due to liquid entering the proximal face 412, causing the central core foam region 446 to push radially outward against the outer shell 448, which may slow down the expansion of the foam implant 400b as a whole. Warm fluid injection on the proximal face 412 may then trigger the outer shell 448 to quickly expand to create an open cell structure on the outside, while the central core foam region 446 expands more slowly to provide placement control.

FIG. 4C illustrates another embodiment similar to that shown in FIG, 4B, but with a flange region 447a on the central core foam region 447, allowing the central core 447 to receive liquid radially into the core 447. The barrier 410c surrounds the central core 447 and separates it from the outer shell 449. The barrier 410c may be impermeable or permeable to liquid.

In all of the above embodiments the barrier 410, 411, 410b, 410c may be an adhesive, polymer filled foam, melted foam structure, closed foam region, polymer film, or metal structure with or without holes extending therethrough. In all of the above embodiments with a plurality of foam regions, the distal most region 444, 448, 449 of foam may directly absorb a large volume of warm liquid from the anatomy or the catheter itself during deployment due to the distal region being exposed to liquid entering the distal end of the delivery catheter containing the foam implant 400a, 400b, 400c. A proximal section of foam 440, 446, 447 in a catheter could directly absorb a moderate volume of liquid from the proximal end of the catheter, generally at a slightly lower temperature from natural cooling or a much lower temperature if cold flushing saline is used. The various regions of any of the foam implants 400a, 400b, 400c described above may be formed from a SMP foam, or they may be formed from any other biocompatible foam.

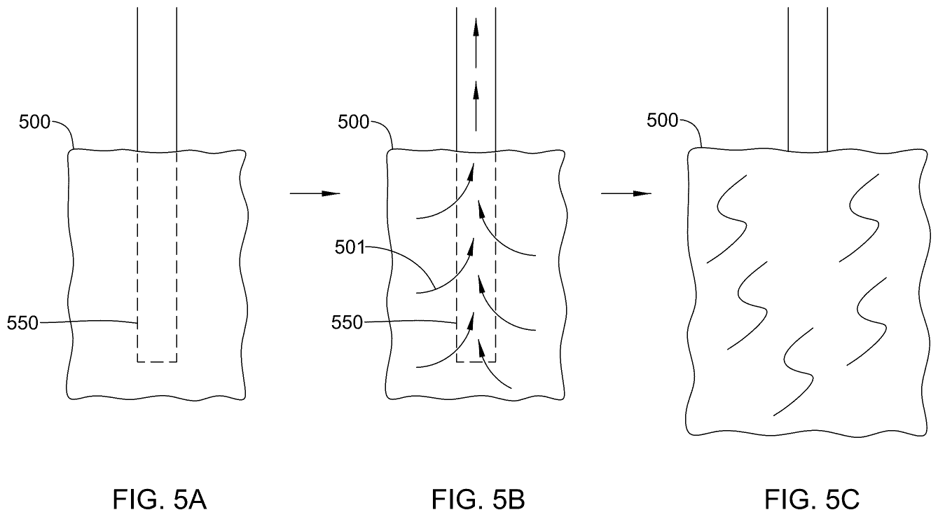

In another embodiment, a crimped SMP foam element 500 may include an internal lumen 550 extending axially through at least a portion of the crimped foam element 500, as shown in FIG. 5A. The internal lumen 550 may be used to drive expansion. In some embodiments, at least a portion of the internal lumen 550 is porous, allowing liquid to flow radially into and out of the lumen. The internal lumen 550 may be formed from a polymer tube inserted into the foam element 500 during formation. When negative pressure is applied to the internal lumen 550, such as by vacuum, liquid may be drawn from outer regions of the foam element 500 and from the environment surrounding the foam element 500 into the internal lumen 550 and out the internal lumen 550, as shown by arrows 501 in FIG. 5B. This application of negative pressure to the internal lumen 550 may improve liquid transfer from outside the foam element 500 into the foam element 500, resulting in expansion of the foam element 500, as shown in FIG. 5C. In some embodiments, the internal lumen 550 may be capped under vacuum to maintain negative pressure within the foam element 500 if a proper seal is achieved. This negative pressure may ensure liquid entering the foam element 500 remains within the foam element to maintain the expanded shape.

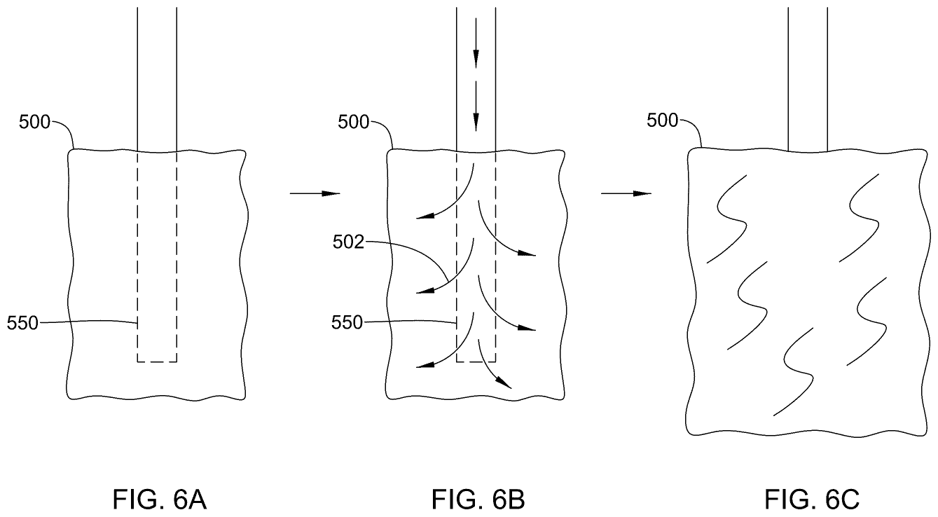

FIGS. 6A-6C illustrate expanding the crimped foam element 500 with the internal lumen 550 by the application of positive pressure to the lumen 550 with liquid. In one embodiment, a liquid solution may be injected through the internal lumen 550 to wet the interior of the foam element 500 via a porous portion of the lumen, either alone or in conjunction with external wetting of the crimped foam element 500. Positive pressure may help drive the liquid radially outward from the lumen 550 through the crimped foam, as shown by arrows 502 in FIG. 6B, improving mass transport compared to passive diffusion. Additionally, contrast solution may be injected into the internal lumen 550, allowing the location of the foam element 500 to be identified.

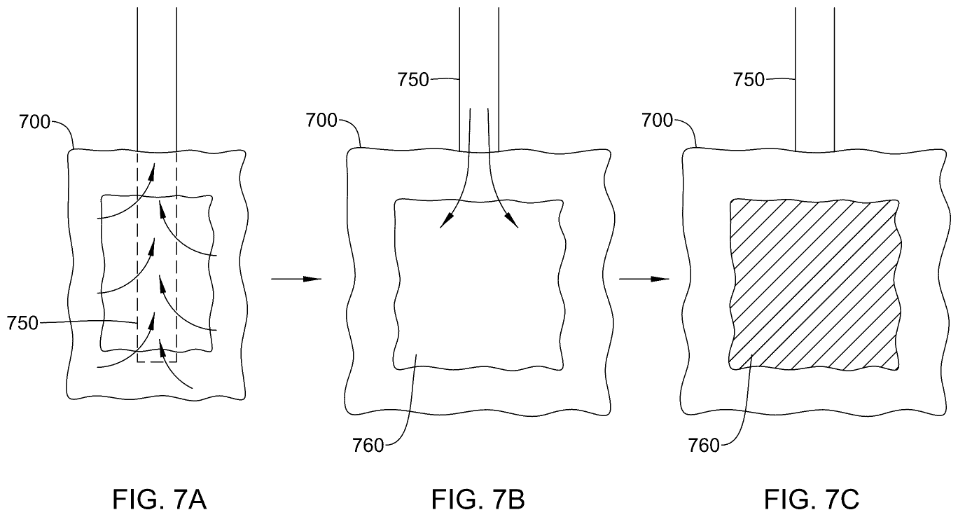

FIGS. 7A-7C illustrate expansion of a further embodiment of SMP foam element 700. Negative pressure applied to a porous central lumen 750 may be used to drive liquid uptake into the foam element 700 from the environment surrounding the foam element 700, as shown in FIG. 7A. In this embodiment, the foam element 700 may have a hollow internal chamber 760, and positive pressure may be used to fill the hollow interior with filler material through the central lumen 750, as shown in FIG. 7B. As shown in FIG. 7C, the entirety of the internal chamber 760 may be filled with filler material which may maintain the foam element 700 in its expanded configuration, regardless of the amount of liquid in the environment surrounding the foam element 700. The mechanical properties of the foam element, including the exterior pore size, may be selected to achieve the desired level of foam element stiffness and structural support. The filler material may be selected to provide additional functionality such as being radiopaque or include one or more drugs to be delivered after implantation and expansion.

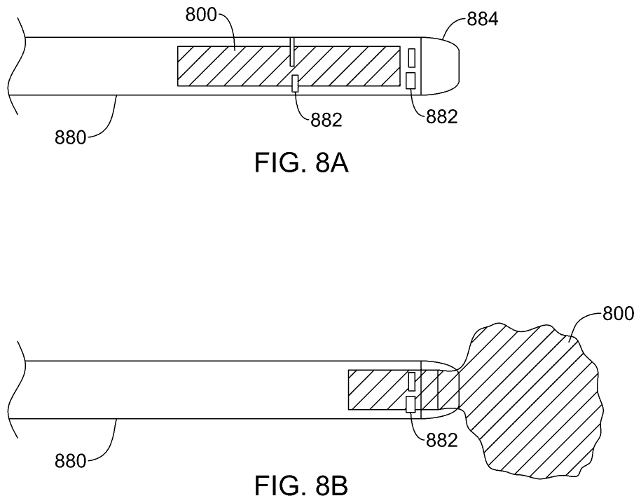

In a further embodiment, the SMP foam element 800 may be configured to remain constrained and captured within a distal end 884 of a delivery catheter 880 while liquid enters the foam element 800 and begins the expansion process. As shown in FIG. 8A, the delivery catheter 880 may have a plurality of openings 882 extending through the side wall of the catheter. In some embodiments, the plurality of openings 882 may be positioned adjacent the distal end 884 of the catheter. In other embodiments, some openings 882 may be positioned further proximal, over the foam element 800 when disposed within the delivery catheter 880. During various procedural steps, liquid may enter the distal end 884 and the openings 882 of the delivery catheter 880 and begin to infiltrate into the foam element 800 to start the expansion process. The procedural steps may include flushing the catheter on the bench prior to insertion, during translation inside an access sheath, or during a dwell time inside the body. When the SMP foam element 800 is delivered through the distal end 884 of the delivery catheter 880 the foam element 800 immediately expands as soon as it clears the catheter, as shown in FIG. 8B.

The method of controlling expansion of a shape memory foam implant, such as the foam element 800 is shown in FIGS. 8A and 8B. The method includes constraining a crimped SMP foam element 800 within a catheter 880 such that only a distal face of the element is exposed, allowing water or other liquid to ingress at the exposed distal face during procedural steps, and fully deploying the foam element 800 from the catheter 880 to achieve apparent instant expansion. The procedural steps may include at least one of flushing, translation through an access sheath, or dwelling within the body. The method may include creating holes 882 in the catheter to allow controlled water ingress prior to full deployment.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a SMP foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the SMP foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the SMP foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the SMP foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D, L-lactide, poly(beta-hydroxybutyrate), polygluconate including poly-D-gluconate, poly-L-gluconate, poly-D, L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the SMP foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An expandable foam implant comprising:
an element formed from a shape memory polymer foam and radially crimped, the radially crimped element having an axial or circumferential outer surface of tightly packed pores resistant to liquid ingress; and
a surface treatment disposed on at least a first portion of the axial or circumferential outer surface, wherein liquid ingress into the first portion is faster than into a remainder of the axial or circumferential outer surface of the radially crimped element.

2. The expandable foam implant of claim 1, wherein the surface treatment includes a plurality of shaved, scored, or abraded regions extending along an outer surface of the crimped element.

3. The expandable foam implant of claim 2, wherein the plurality of shaved, scored, or abraded regions includes a plurality of axially extending abraded stripes spaced apart around a circumference of the crimped element.

4. The expandable foam implant of claim 3, comprising 15-20 axially extending abraded stripes each measuring 1.0 mm to 2.0 mm in a circumferential direction.

5. The expandable foam implant of claim 2, wherein the plurality of shaved, scored, or abraded regions includes a plurality of helical grooves.

6. The expandable foam implant of claim 2, wherein the plurality of shaved, scored, or abraded regions cover between 25% and 50% of the axial or circumferential outer surface.

7. The expandable foam implant of claim 1, wherein the surface treatment includes a temporary agent disposed within pores in the foam before being radially crimped, the temporary agent configured to be dissolved when the element is exposed to liquid.

8. The expandable foam implant of claim 7, wherein the temporary agent includes at least one of a polymer, salt, or sugar.

9. The expandable foam implant of claim 7, wherein areas of the foam devoid of the temporary agent expand before areas with the temporary agent.

10. The expandable foam implant of claim 7, wherein the temporary agent has a removal time that is tuned to achieve a desired expansion profile.

11. The expandable foam implant of claim 7, wherein areas of the element with the temporary agent expand more quickly than areas without the temporary agent when the temporary agent has a short removal time.

12. The expandable foam implant of claim 1, further comprising an internal permeable lumen within the crimped element.

13. The expandable foam implant of claim 12, wherein the internal permeable lumen is configured to allow application of negative pressure to draw liquid into the element or positive pressure to inject liquid directly into the interior of the element.

14. The expandable foam implant of claim 12, wherein the internal permeable lumen is formed by a permeable polymer tube.

15. An expandable foam implant comprising:
an element including at least first and second regions of open-celled foam; and
at least a first barrier region separating the first and second regions, the first barrier configured to restrict liquid flowing between the first and second regions;
wherein liquid ingress into the first region is faster than into the second region.

16. The expandable foam implant of claim 15, wherein the first region is a cylindrical shell and the second region is a cylindrical core surrounded by the first region.

17. The expandable foam implant of claim 15, further comprising a third region, wherein the first region defines a distal end of the element, the second region defines a middle region of the element, and the third region defines a proximal region of the element, wherein a first barrier is disposed between the first and second regions and a second barrier is disposed between the second and third regions, wherein the first region is configured to expand first when the element is exposed to liquid, followed by the second region, with the third region expanding last.

18. A method of controlling expansion of a shape memory foam implant, comprising:
constraining a crimped shape memory foam element within a catheter such that only a distal face of the element is exposed;
allowing liquid ingress to occur at the exposed distal face during procedural steps; and
element from the catheter to achieve apparent instant expansion.

19. The method of claim 18, wherein the procedural steps comprise at least one of flushing, translation through an access sheath, or dwelling within a body.

20. The method of claim 18, further comprising creating holes in the catheter to allow controlled liquid ingress prior to full deployment.
